(19) United States
(12) Patent Application Publication
(10) Pub. No.: US 2024/0371202 A1
(43) Pub. Date: Nov. 7, 2024

(54) SYSTEM AND METHOD FOR PREDICTING FACIAL BIOLOGICAL AGE BASED ON METHYLATION MARKERS AND FACE IMAGE DATA

(71) Applicant: Ali Mostashari, New York, NY (US)

(72) Inventors: Ali Mostashari, New York, NY (US); Raya Khanin, New York, NY (US); Klemo Vladimir, New York, NY (US); Mario Storga, New York, NY (US)

(73) Assignee: LifeNome Inc., New York, NY (US)

(21) Appl. No.: 18/140,014

(22) Filed: Apr. 27, 2023

Related U.S. Application Data
(60) Provisional application No. 63/453,332, filed on Mar. 20, 2023.

(51) Int. Cl. G06V 40/16

(52) U.S. Cl. CPC G06V 40/178; G06V 40/171

(57) ABSTRACT

A method of predicting facial biological age of an individual is provided comprising a computer receiving methylation data and facial image data describing an individual. The method also comprises the computer receiving survey data provided by the individual and the computer applying an age predictor clock model to at least the received data to predict a facial biological age of the individual. The method also comprises the computer identifying causal methylation markers in the methylation data. The method also comprises the computer generating a personalized report for the individual describing the predicted facial biological age based on methylation markers, and the report further describing methylation markers causal to facial aging, the markers identified at least in the data. The personalized report further contains facial ageotypes from face image data. The age predictor clock model is trained and validated on reference population data stored in the reference population database.

### CROSS REFERENCE TO RELATED APPLICATIONS

The present non-provisional patent application is related to U.S. Provisional Patent Application No. 63/453,332 filed Mar. 20, 2023, the contents of which are incorporated herein in their entirety.

### FIELD OF THE DISCLOSURE

The present disclosure is in the field of determining facial biological ages using epigenetic markers such as the methylation status of nucleotides in the genomic DNA from a biological sample (CpGs). More particularly, the present disclosure provides systems and methods for identifying CpG markers causal to facial aging, utilizing the markers in developing an accurate age predictor using machine learning, and computing the facial biological age of a subject individual. Embodiments of the present disclosure provide a platform that integrates methylation data, face image data, and survey data to update computations of facial biological age.

### BACKGROUND

Aging is a multifactorial process. People age at different rates. It has been demonstrated that chronological age may not always reflect the biological age of a given person. Hence, numerous biological clocks based on methylation markers, and other high-throughput biomarkers such as gene expression, protein levels, and microbiota, have been proposed to measure the biological age of a person. Biological clocks are developed using machine learning models on a training dataset where the chronological age of a sample is known and further validated on a different dataset.

DNA methylation has become a widely accepted standard biomarker for human age estimation. A first-generation of epigenetic clock was initially developed using large datasets of samples to predict chronological age. Some biological clocks use methylation data from blood samples.

Other biological clocks are focused on specific tissues, including skin. A very common approach for clock building is to use a ‘correlation-with-age’ method, where CpGs with higher correlations/anti-correlations with age are given more predictive power in the age-predictor model. (Reference 1)

Growing evidence indicates that such epigenetic clocks capture biological aspects of aging. The clocks further have high accuracy in predicting a person's biological age compared to the population data.

Second and third-generation biological clocks have been purposely developed to predict biological age and mortality risk. (Ref. 2) These clocks demonstrate consistent associations with all-cause mortality, age-related clinical phenotypes, and cognitive performance measures. (Ref. 3)

Biological clocks are widely utilized in the burgeoning anti-aging and longevity industries for determining biological ages of samples and more recently for testing efficacy of anti-aging treatments. However, biological clocks are generally built on CpGs that are purely correlated with chronological age or age-related conditions.

Biological clocks built on CpGs should therefore be used with caution. It is not clear whether the CpG markers that constitute the features of biological clocks are molecular drivers of aging or are in fact consequences of aging processes. Hence, CpGs utilized in epigenetic clocks do not necessarily reflect underlying age-related biological processes. Consequently, an objective is to identify CpGs that are early modifiable biomarkers of aging that occur upstream of critical age-related processes.

Facial aging is a shared aesthetic concern worldwide. Many patients seek non-invasive cosmetic procedures for the management or prevention of cutaneous manifestations of aging, with over 83% of patients motivated by the idea of achieving a more youthful appearance. (Ref. 4)

Facial aging is a multifactorial process that can be partitioned into intrinsic and extrinsic subprocesses, each with overlapping and distinctive histological hallmarks that have long been acknowledged by the antiaging cosmetics industry. Intrinsic aging is the natural aging process of the skin and is determined by internal factors, including genetics. Photoaging (extrinsic aging) is accelerated aging of the skin caused by exposure to environmental factors such as pollution, sun exposure, and climate. Age-related skin changes may be attributed to combinations of intrinsic and extrinsic aging as well as lifestyle factors, exercise, stress, sleep, nutrition, and skincare routine.

Facial skin changes are the most evident indicators of aging manifesting symptoms (ageotypes) such as dehydration, loss of skin elasticity and firmness, laxity, wrinkles, photodamage, uneven pigmentation, solar lentigines, solar elastosis, inflammation, glycation, rough-textured appearance, actinic keratosis, skin cancers (keratinocyte cancer, basal cell carcinoma, melanoma), and other age-related skin diseases and conditions. At molecular and cellular levels, multiple interconnected biological pathways, genetic programs, and epigenetic modifications underlie facial aging processes.

The very large antiaging cosmetic industry has primarily been focused on discovering the most effective products that conceal or temporarily mitigate the visible downstream effects of aging, rather than proactively addressing the upstream root causes of aging. (Ref. 5)

Facial ageotypes such as skin elasticity, firmness, tightness, laxity, dehydration, photodamage, wrinkles, and others, are assessed either subjectively, by a health or beauty professional. More recently, ageotypes may be assessed by image processing devices, or smartphone cameras, when the ageotypes are readily observable and visible by either the naked eye or an imaging device.

There is hence a large unmet need for non-invasive systems, methods, and devices that are capable of accurately predicting facial biological age based on early and modifiable biological markers, such as DNA methylation markers (CpGs) and other available information. Further, there is a lack of aging clocks purposely built to accurately predict facial biological age by capturing facial age-related changes (facial ageotypes). Identification of causal epigenetic biomarkers that are not only correlated with age but reflect damage accumulated with age, or protection from aging, will enable the development of preventative anti-aging measures that alter or even reverse causal epigenetic biomarkers.

### PRIOR ART

Driven in part by the large size of the antiaging cosmetics industry, there is intensive research is ongoing that addresses age-related skin changes. However, only a few previous implementations address prediction of facial and skin biological age using methylation markers.

Several prior disclosures claim to determine the age of a biological sample using a plurality of DNA methylation markers. An earlier disclosure is US Publication 2016-0222448, entitled “Method to estimate the age of tissues and cell types based on epigenetic markers.” This disclosure claims a method for determining the age of a biological sample using a linear combination of the methylation status of nucleotides (e.g., CpG) in the genomic DNA. This multi-tissue age predictor (DNAm Age) is based on 354 CpGs that are purely statistically correlated with chronological age in multiple tissue samples. A principal shortcoming of US Publication 2016/0222448 is that it uses CpGs that are purely correlated with age and may not be reflective of underlying age-related processes.

Another earlier disclosure, US Publication 2020/0190568A1, entitled “Methods for detecting the age of biological samples using methylation markers” utilizes CpGs to estimate the age of a skin sample. While this disclosure predicts age with high accuracy, the disclosure utilizes CpGs that are purely correlated with chronological age and thus may not reflect age-related processes in the skin.

Shortcomings therefore exist in previous implementations regarding systems and methods that predict skin aging based on methylation markers. Some previous implementations do not identify CpGs that are causal to facial aging and do not address how to distinguish between CpGs that are driving aging from those that are consequences of aging. Previous implementations also do not integrate methylation markers with other data such as imaging or survey data.

### BRIEF DESCRIPTION OF THE FIGURES

FIG. 1 is a diagram illustrating modules of a system according to an embodiment of the present disclosure.

FIG. 2 is a block diagram of a system according to an embodiment of the present disclosure.

FIG. 3 is a chart listing CpG markers significantly associated with facial aging according to an embodiment of the present disclosure.

### DETAILED DESCRIPTION

Systems and methods provided herein address shortcomings of previous implementations and claim a dynamic self-learning system to infer DNA methylation markers (CpGs) causal to facial aging. Systems and methods provided herein further build an accurate facial biological age predictor, using a machine learning model, that is trained and validated on dynamically updated methylation data that is integrated with face image data and survey data.

Systems and methods provided herein analyze DNA methylation markers for individual persons, integrate the markers with face image data and other information from surveys and feedback, and predict, using machine learning methodology, facial biological skin age for subject individuals. The present disclosure provides systems and methods for identifying DNA methylation markers that are causal to facial aging.

Systems and methods provided herein predict the facial biological age of persons based on methylation data, face image data, and survey data. A principal objective herein is to improve the assessment of the facial biological age and elucidate causal methylation markers as early and modifiable signs of facial aging.

A platform is provided herein for collecting large amounts of heterogeneous data from individuals that may provide bases for longitudinal studies. In embodiments, the platform develops novel anti-aging compounds and treatments. The platform also assesses efficacies of anti-aging treatments and compounds by comparing facial biological age before and after treatment for a general population, or for specific population groups within a general population.

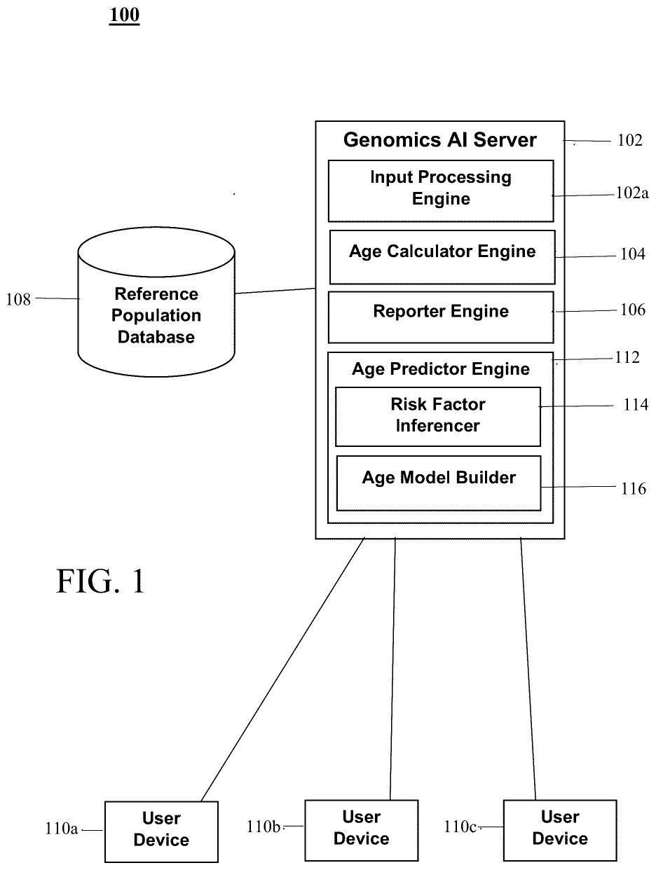

Turning to the figures, FIG. 1 illustrates components and interactions of a system 100 for predicting facial biological age. As depicted, the system 100 comprises a genomics AI server 102 that comprises an input processing engine 102a, an age calculator engine 104, a reporter engine 106, an age predictor engine 112, and a reference population database 108.

The system 100 also comprises a plurality of user devices 110a-c used by individuals to submit data via the input processing engine 102a to the genomics AI server 102 and to receive personalized reports and other data from the genomics AI server 102 via the reporter engine 106 and other components. The age predictor engine 112 comprises a risk factor inferencer 114 and an age model builder 116. While quantity three user devices 110a-c are depicted in FIG. 1 and provided by the system 100, in embodiments more than or less than quantity three user devices 110a-c may be provided.

The genomics AI server 102 may be a single computer or multiple physical computers situated at one or multiple geographic locations. While the input processing engine 102a, the age calculator engine 104, the reporter engine 106, and the age predictor engine 112 are depicted in FIG. 1 as contained by or components of the genomics AI server 102 and executing on the genomics AI server 102, in embodiments the input processing engine 102a, the age calculator engine 104, the reporter engine 106, and the age predictor engine 112 may be separate components or software executing on separate devices proximate or remote from the genomics AI server 102.

While referred to as engines, the input processing engine 102a, the age calculator engine 104, the reporter engine 106, and the age predictor engine 112 may be combinations of hardware and software applications or entirely software applications. Components described herein as modules, submodules, or devices may be physical devices, combinations of a physical device and software, or entirely software. For example, a risk factor inferencer module 114 and an age model builder module 116 may be combinations of hardware and software or primarily software.

The genomics AI server 102 receives methylation data, face image data, and survey data from individuals using the user devices 110a-c. The received data is processed by the input processing device 102a of the genomics AI server 102 and stored in the reference population database 108. The received data is also provided to the age calculator engine 104 to compute a facial biological age for an individual by applying an age predictor clock model trained and validated on the reference population data in the age predictor engine 112.

Based on the facial biological age calculated by the age calculator engine 104, the reporter engine 106 generates a personalized report for the subject individual with a predicted facial biological age based on methylation markers and methylation markers causal to facial aging, identified in the individual's sample. The personalized report may further contain facial ageotypes from face image data.

The personalized report may further contain a comparison of the individual's data with the reference population data and contain comparisons of the individual's data at different times. The personalized report may further be utilized by the individual, or third-party, for example, a dermatologist or beauty professional, for recommending skincare products and treatments.

Feedback may be provided that collects data at a later time via a survey questionnaire, and/or face image device and transmits the data back to the reporter engine 106 and the reference population database 108. Additional methylation data may later be collected from the individual and transmitted to the reference population database 108. Data collected at least via feedback is utilized to build a longitudinal data platform for improving facial biological prediction and identifying causal methylation markers for facial ageotypes.

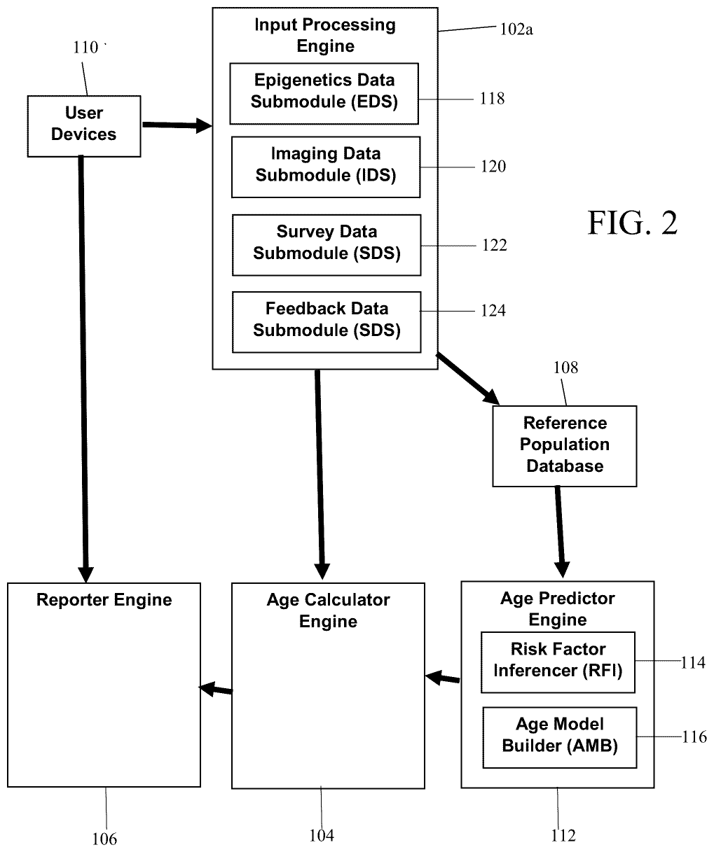

FIG. 2 is a block diagram that illustrates details of an embodiment including the input processing engine, reference (population) storage, age calculator engine, age predictor engine, and reporter engine. The input processing engine 102a receives epigenetics data, face image data, and other information from a subject via user devices. The input processing engine 102a consists of four submodules: an epigenetics data submodule (EDS) 118, an imaging data submodule (IDS) 120, a survey data submodule (SDS) 122, and a feedback data submodule (FDS) 124. In some embodiments, data input is provided via a web, or mobile application at home, or in a professional environment at a healthcare provider, or beauty provider facility.

The input processing engine 102a receives and processes epigenetics data from various sources via the epigenetics data submodule 118 that may be integrated with external information providers or databases. In some embodiments, epigenetics input data may be a file that contains DNA methylation markers (CpGs) uploaded by an individual, uploaded by an external genotyping or sequencing service/company using a generic or proprietary application programming interface (API), or uploaded by a third party, for example, physicians, aestheticians, or beauty services provider. In embodiments, DNA methylation markers (CpGs) are pre-processed using appropriate bioinformatics methods directed to obtaining quantifiable results to enable further assessments.

The input processing engine 102a receives and processes face image data from various sources via imaging data submodule (IDS) 120. Face image data is taken by smartphones, professional devices, photobooth analyzers, and other relevant devices.

The IDS 120, which may be partially integrated with external information providers and databases, enables input of face image data by generic or proprietary API from imaging devices. In preferred embodiments, the IDS 120 enables input of processed face image data by extracting features from face image by a proprietary, open source, or third-party algorithm utilized via API from relevant imaging devices.

The input processing engine 102a receives survey data from various sources via the survey data submodule (SDS) 122. Survey data includes at least chronological age and sex. In an embodiment, survey data includes perceived facial age as reported by a third party, or self-reported.

Survey data, collected via at least questionnaire, may further include questions directed to at least one of skin type (dry, normal, oily, combination, sensitive); Fitzpatrick skin tone scale; skin concerns (e.g. dehydrated, acne prone, undereye bags), skin conditions (eczema, acne, rosacea, psoriasis), skin aging (e.g. pigmented spots, sagging eyelids). Survey data, collected via the questionnaire, may further include geolocation, demographics, environment, diet (including allergies, sensitivities, restrictions, and preferences), physical activity level, and general health concerns. The survey data submodule (SDS) 122 enables integration with self-reported questionnaires or data input by third parties.

The feedback data submodule (FDS) 124 is utilized when the user provides feedback regarding the personalized report. In preferred embodiments, the FDS 124 enables input of methylation data to compare the facial biological age of an individual before and after a recommended skincare treatment. In preferred embodiments, the FDS 124 enables input of face image data to compare with facial image data before a recommended skincare treatment.

The FDS 124 also receives reviews, survey responses, or other feedback from the individual about specific skincare ingredients, skincare products, or skincare regimens, and likes/dislikes. The FDS 124 may be used by the user, or a third party (e.g. healthcare or beauty professional) to report adverse reactions to specific skincare ingredients or products such as irritations, allergies, or sensitivities.

Upon receipt of methylation data, face image data, and survey data, the input processing engine 102a propagates the received data to the reference population storage 108 which is a repository of methylation, face image, and survey data for a plurality of individuals. The data stored in the reference population storage 108 is continuously updated with new entries received from individuals via the input processing engine 102a. Reference population storage 108 can also be updated by bulk downloads of methylation data from multiple individuals and from public repositories of methylation data, as well as from face imaging data from external sources, data repositories, and third parties.

Feedback data, received from the user or third-party, is propagated to the reference population storage 108. After processing, using suitable data analysis tools, the feedback data is further propagated to the age predictor engine 112 and reporter engine 106 to further improve a facial biological age prediction algorithm and identify methylation markers that are either causal drivers of facial aging or causal anti-aging methylation markers.

A continuous self-learning system may thereby be set into place. For example, by analyzing, via the age predictor engine 112, collected data in the reference population storage 108, the system improves predictions for the facial biological age. The system may further build predictive models for other facial age-related phenotypes (e.g. wrinkles, pigmented spots), skin age-related conditions (e.g. seborrheic keratosis), and skin diseases (e.g. basal cell carcinoma).

The system may infer that individuals with specific combinations of methylation markers are more likely to develop premature wrinkles, in particular, if they live in polluted areas. Such individuals may have CpGs that affect expression levels of the elastin ELN gene, and/or elastin protein, or collagen-producing genes. Similarly, the system may learn that specific skincare ingredients (e.g. resveratrol), and lifestyle changes (e.g. high-level SPF) may assist high-risk individuals in delaying the development of premature wrinkles.

The reference population storage 108 provides a basis for updating, via a machine learning methodology, the age predictor engine 112. Further, the reference population storage 108 may provide a basis for generating a personalized report performed in the reporter engine 106.

The age predictor engine 112 comprises two modules: a risk factor inferencer (RFI) 114 module and an age model builder (AMB) module 116. The risk factor inferencer 114 module identifies, by applying epigenome-wide Mendelian Randomization (EWMR), methylation markers causal to facial aging and facial skin age-related phenotypes (ageotypes). The risk factor inferencer module 114 further validates identified methylation markers using the data from the reference population database 108.

Mendelian randomization (MR) is an established genetic computational approach for causal inference that recapitulates the principle of a randomized clinical trial (RCT) as it utilizes genetic variants as instrumental variables. While RCTs generally consider the effect of treatment (exposure) by comparing the cases and the controls, the MR uses the genetic variants (SNPs) that are robustly associated with the exposure as instrumental variables as SNPs are randomly assigned at conception and therefore are not biased by environmental confounders. Hence, MR is used as a computational tool for investigating causal relationships between DNA methylation, as exposure, and facial skin aging or facial skin ageotypes as outcomes.

In an embodiment, epigenome-wide MR utilizes summary statistics data from a genome-wide association study (GWAS) on perceived facial age as an outcome. EWMR further utilizes as an exposure a publicly available dataset that has 11,165,559 SNP-CpG associations (meQTLs; P<10-14, whole blood samples) identified through GWAS from 6994 samples. (Ref. 6)

In the embodiment introduced immediately above, the EWMR yields 1299 novel methylation markers (CpGs) causal to perceived facial age (p<=0.001). Some of the top causal CpG sites are within genes with roles in skin pigmentation (IRF4, ASIP, BCN2, MC1R) that also play roles in skin aging. (Ref. 7)

Further, many causal CpG sites are within genes coding for extracellular matrix (ECM) proteins that drive structural changes in the skin during aging causing loss of skin elasticity and wrinkles. For example, a CpG (cg05010648) in the ELN gene coding for the elastin protein has a large negative effect on perceived facial aging (OR=0.59). This CpG is protective from aging. Several CpGs in two collagen biosynthesis genes COL1A1 and COL8A1 are also protective from aging.

Extending the analysis to the protein space via epigenome-wide association studies (EWAS) catalog points to several additional age-protective CpGs that causally increase levels of collagen proteins (COL2A1, COL3A1). These findings indicate that some methylation markers, identified in whole blood data, using EWMR, are age-protective as they may causally slow down skin aging via increasing levels of collagen genes and proteins.

Other examples related to collagens include an age-damaging CpG that causally lowers levels of another collagen protein (COL6A1) and an age-damaging CpG (cg13280184) that causally increases levels of the protein MMP3 which is known to be upregulated with age. The MMP3 enzyme degrades ECM proteins, such as type IV, V, IX, and X collagens, gelatin, fibrillin-1, fibronectin, laminin, and proteoglycans, contributing to photoaging, wrinkle formation, and skin laxity. Thus, other CpGs, identified in whole blood, using EWMR, are causally damaging as they increase visible signs of aging.

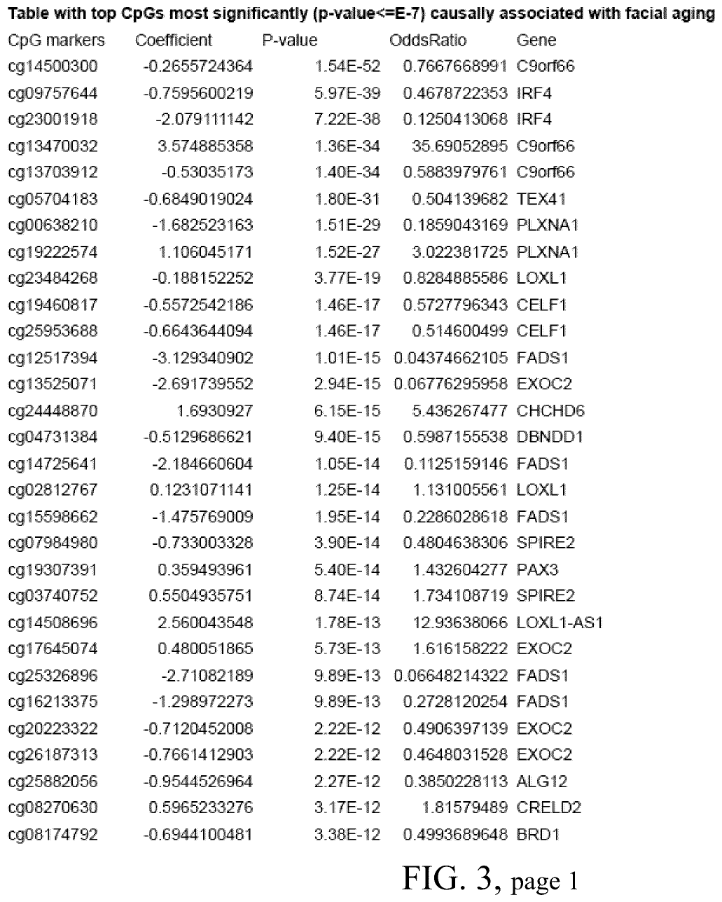

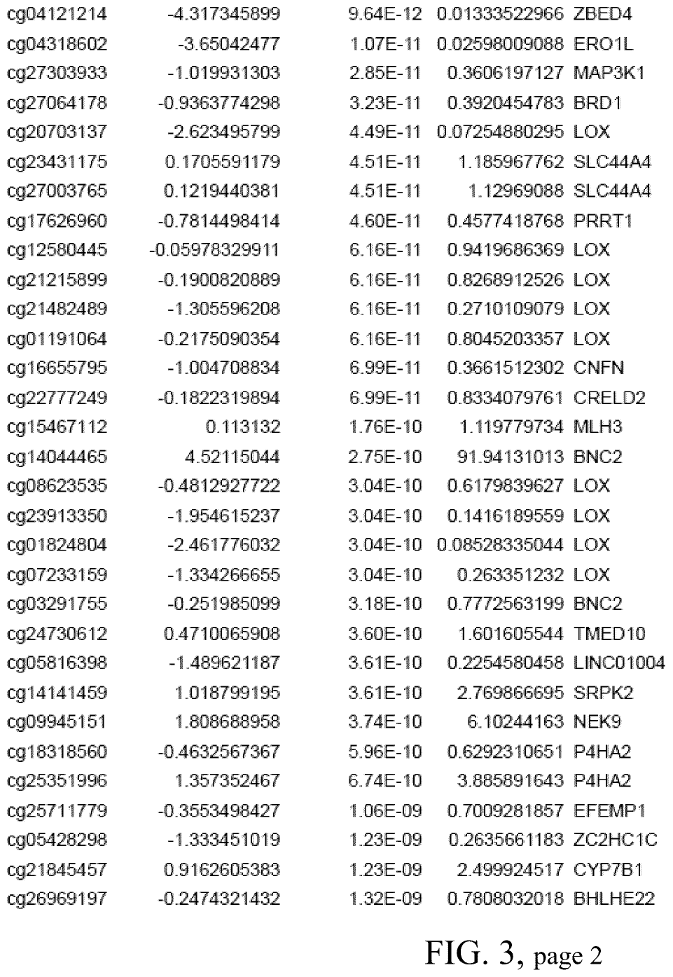

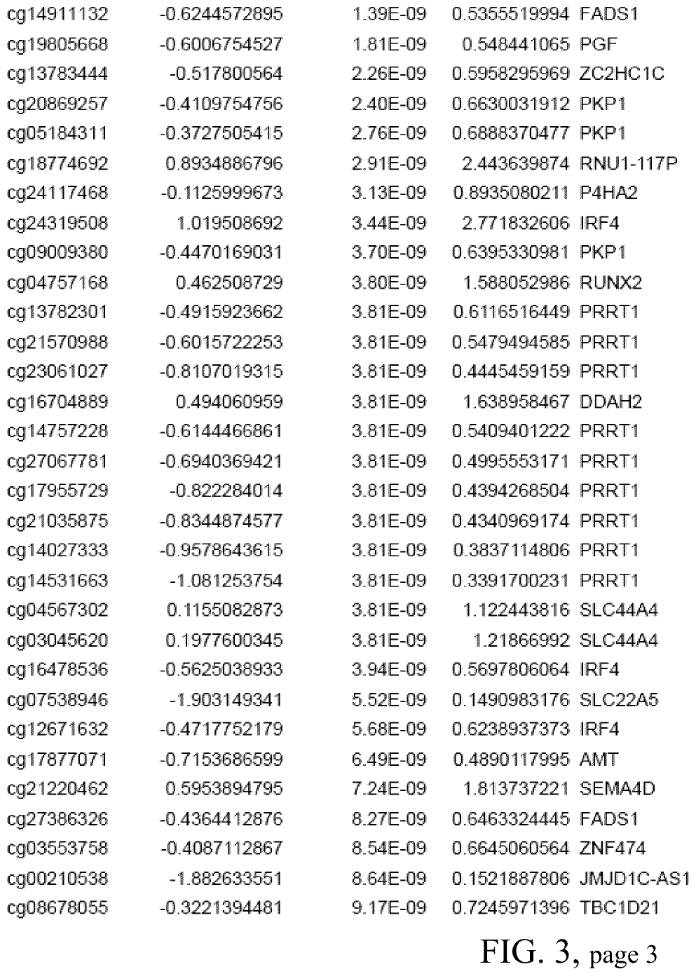

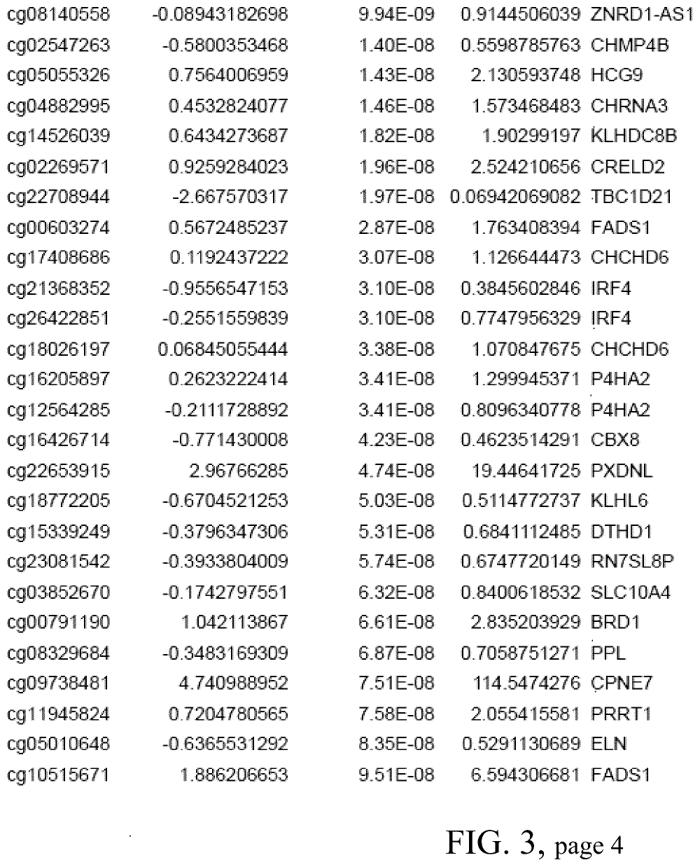

These findings demonstrate that CpG sites causal to perceived facial aging can be identified from the whole blood data. Indeed, earlier studies reported significant overlaps of cis meQTLs (45-73%) and targeted CpG sites (31-68%) among tissues with consistent signs of cis-acting effects across tissues (Ref. 8). Hence, CpGs causal to facial aging, identified in whole blood, may assist in elucidating biological mechanisms underlying skin aging, and validating the efficacy of topical and ingestible anti-aging compounds. FIG. 3 is a chart listing CpG markers significantly associated with facial aging according to an embodiment of the present disclosure.

In other embodiments, other meQTL datasets, publicly available or proprietary, can be utilized as an exposure in EWMR. An example is the GoDMC meQTL dataset that contains SNP-CpG associations for 420,509 CpG sites identified in whole blood samples from 27,750 subjects. In other embodiments, facial age-related phenotypes such as skin pigmentation traits (pigmented spots, photodamage grading), skin cancer traits (seborrheic keratosis, actinic keratosis, basal cell carcinoma), telangiectasia, global wrinkling score, telomere length can be used as outcomes EWMR to infer causal methylation markers of facial aging.

Mendelian Randomization is in some embodiments a two-sample Mendelian Randomization that utilizes linear regression. In some embodiments, Mendelian Randomization is three-sample Mendelian Randomization. In some embodiments, Mendelian Randomization utilizes non-linear models between exposure and outcome. A person skilled in the art appreciates that various models, linear and non-linear, can be built between exposures and outcomes to infer causal methylation markers.

The age model builder 116 develops, via a supervised machine learning methodology, a predictive model for facial biological age using data from the reference population storage 108. In the preferred embodiment, CpGs causal to facial aging are utilized as input features. The age model builder 116 may perform the computations of predictive age model models using at least one algorithm that may be proprietary and/or developed by a third-party source and following best practices of machine learning.

The age model builder 116 is trained on a training dataset that consists of human epidermal, dermal, or whole skin samples, each sample having more than 450,000 methylation probes (CpG) and additional information that includes at least age, tissue type, and sex. The age predictor is validated on an independent dataset of human epidermal, dermal, or whole skin samples. In a specific illustration, the training and validation datasets include three publicly available datasets GSE51954, GSE90124, E-MTAB-4385, each comprising methylation data, and additional information such as age, sex, and tissue type.

In preferred embodiments, the training and validation datasets include proprietary data. Publicly available datasets are merged with proprietary data, pre-processed, and normalized using bioinformatics methods. Datasets are divided into training and validation so that they are age-balanced, sex-balanced, and tissue-balanced (epidermis, dermis, whole skin).

In the preferred embodiment, the age model is learned using an elastic net model using, as input features, CpG markers causal to perceived facial age as identified by EWMR in the risk inference module. In other embodiments, the age predictor is learned using an elastic net model using, as features, CpG markers that are significantly associated with facial ageotypes extracted from face image data, or survey data.

In a specific embodiment, the elastic net model includes the CpG feature-specific penalty factor informed by the causality rank that is based on at least one of the causal effect sizes and p-values from EWMR analyses or a quantitative measure computed by a bioinformatics tool (e.g. colocalization probability). In other embodiments, the age predictor is learned by another supervised machine learning algorithm, wherein CpG features are ranked by taking the causality rank into account. In other embodiment, CpG features are ranked by a quantitative measure based on correlations with facial ageotypes extracted from face image data, or survey data.

The age calculator engine 104 is a computing device that receives methylation data from an individual via the input processing engine 102a. It further receives causal methylation markers and the age predictor model from the age predictor engine 112. The age calculator engine 104 further identifies causal methylation markers in the individual's methylation data and calculates the facial biological age of the individual using the age predictor model.

The reporter engine 106 receives causal methylation markers and facial biological age for an individual from the age calculator engine 104 and generates a personalized report informing individuals about their facial biological age, age-related damaging methylation markers, and age-protective methylation markers. In other embodiments, specific skincare treatments and ingredients can be identified, by computational analyses, as reversing the effect of specific age-damaging methylation markers or improving age-protective methylation markers.

Feedback provided by individuals is done via user devices 110 that collect data and responses from individuals on the provided, by the system, personalized reports. The feedback data may comprise methylation data collected from individuals at different time-points. The feedback data may further comprise face image data, and data from survey questionnaires. The feedback responses may comprise questionnaires on comprehension of information provided by personalized reports.

Additionally, the feedback responses may comprise liking/disliking recommendations of skincare ingredients, products, and treatments. The feedback data may then be transmitted to the reference population database 108 by using FDS 124, and, after processing, be further transmitted to the age predictor engine 112 to improve facial biological age prediction algorithms. A continuous self-learning system may thereby be set in place. The feedback responses may be transmitted to the reporter engine 106 to improve personalized reports.

In some embodiments, the user devices 110 a-c may be mobile computing devices such as a smartphone or a tablet computing device. In some embodiments, the user devices 110 a-c may be a desktop computing device or a laptop computing device. In some embodiments, the user devices 110 a-c may include more than one computing device, such as a user computing device configured to provide a user interface and one or more server computing devices configured to provide computational functionality. In such embodiments, the user computing device and one or more server computing devices may communicate via any suitable communication technology or technologies, such as a wired technology (including but not limited to Ethernet, USB, or the Internet) or a wireless technology (including but not limited to WiFi, WiMAX, 3G, 4G, LTE, or Bluetooth).

In an embodiment, a method of predicting facial biological age of an individual is provided. The method comprises a computer receiving methylation data describing an individual and receiving facial image data describing the individual. The method also comprises the computer receiving survey data provided by the individual and the computer applying an age predictor clock model to at least the received data to predict a facial biological age of the individual.

The method also comprises the computer identifying causal methylation markers in the methylation data. The method also comprises the computer generating a personalized report for the individual, the report describing the predicted facial biological age based on methylation markers, and the report further describing methylation markers causal to facial aging, the markers identified at least in the data. The personalized report further contains facial ageotypes from face image data.

The method also comprises the computer copying the received data and the predicted facial biological age to a reference population database. The age predictor clock model is trained and validated on reference population data stored in the reference population database.

Face image data of the individual is one of a selfie image taken by a smartphone camera and an image captured by a professional imaging device. Facial age-related phenotypes (ageotypes) are extracted, via a machine learning (AI) classifier, from the facial image data, wherein the classifier is one of a proprietary, an open-source, and a third-party algorithm utilized via an application programming interface (API).

In another embodiment, a system for continual improvement of age prediction based at least on methylation data is provided comprising a computer and application executing thereon that receives epigenetics data containing at least DNA methylation markers describing an individual. The system also receives facial image data describing the individual and receives feedback data and survey data comprising at least chronological age and gender of the individual. The system also predicts a facial biological age of the individual based on the data and propagates the received data and the predicted age to a reference population storage.

The system uses the received data and previously stored data to improve a facial biological age prediction algorithm. The feedback data is further propagated to an age predictor engine and a reporter engine to improve a facial biological age prediction algorithm and identify methylation markers that are one of causal drivers of facial aging and causal anti-aging methylation markers.

DNA methylation markers (CpGs) are pre-processed using bioinformatics methods directed to obtaining quantifiable results to enable further assessments. The system enables input of methylation data to compare facial biological ages of individuals before and after a recommended skincare treatment provided by at least a third party. The system builds predictive models for facial age-related phenotypes comprising at least one of wrinkles and pigmented spots, for skin age-related conditions comprising at least seborrheic keratosis, and for skin diseases comprising at least basal cell carcinoma.

In yet another embodiment, a method for using methylation markers associated with ageotypes is provided comprising a computer applying epigenome-wide Mendelian Randomization (EWMR) to received data describing at least one individual. The method also comprises the computer identifying, via the applied EWMR, methylation markers (CpGs) causal to at least one ageotype. The method also comprises the computer utilizing epigenome-wide methylation (meQTL) data as exposure and the computer validating the identified methylation markers.

The method also comprises the computer validating markers using data from a reference population database. The method also comprises the computer applying the EWMR to utilize summary statistics from genome-wide association studies for facial ageotypes as outcomes.

The method also comprises the computer observing and measuring facial ageotype and extracting ageotype from at least one of face image data, survey, and feedback data. Epigenome-wide methylation data (meQTL) contain SNP-CpG associations detected in a biological sample comprising at least one of whole blood, skin, hair, and saliva.

Methylation markers (CpGs) associated with at least one facial ageotype are identified by one of correlative analyses and generalized linear regression from reference population data. Facial ageotype data are at least one of observable and measurable and are extracted from at least one of face image data, survey data, and feedback data.

Steps of systems and methods provided herein may be as follows:

1. The system receives an individual person's epigenetic data (DNA methylation markers, CpGs).
2. The system adds the individual's epigenetic data to population data and compares the individual's data to the reference population data.
3. The system further receives imaging data of the individual's face, via a selfie, digital camera, imaging device, or photobooth analyzer (face image data).
4. The system adds the individual's face image data to population face image data, compares the individual's face image data to the reference population face image data, and integrates population epigenetics data with population face image data.
5. The system further receives survey data from the individual, wherein the data can be self-reported, or collected by a healthcare provider, or beauty professional. Survey data includes at least an individual's age and sex, and it may further include information on skin health, skincare regimen, and lifestyle data.
6. The system adds the individual's survey data to the reference population survey data, compares the individual's survey data to the reference population survey data, and integrates population epigenetics data with population face image data and population survey data.
7. The system collects longitudinal data that includes epigenetic data, face image data, and survey data from a plurality of individuals measured at various time intervals. Survey data may include feedback on skincare recommendations, ingredients, and products including liking/disliking, subjective assessments, and adverse effects. Survey data may be self-reported or reported by a third party.
8. The system propagates the individual's longitudinal data to storage with population data.
9. The system computes facial biological age for the individual by utilizing an age predictor model using a plurality of methylation markers identified in the methylation data of the individual.
10. The system generates a personalized report that contains the individual's predicted facial biological age, age-related methylation markers, and facial ageotypes from face image data.
11. The system relies on a reporting and feedback module to send and receive the material.

### REFERENCES

1) Li et al. (2022) Novel feature selection methods for construction of accurate epigenetic clocks. PLOS Comput Biol 18 (8)
2) Lu et al. DNA Methylation GrimAge Strongly Predicts Lifespan and Healthspan. Aging 2019, 11, 303-327; Bell et al. DNA Methylation Aging Clocks: Challenges and Recommendations. Genome Biol. 2019, 20, 249
3) Duan et al. Epigenetic Clock: A Promising Biomarker and Practical Tool in Aging. Ageing Res. Rev. 2022, 81, 101743; Gialluisi et al. Epidemiological and Genetic Overlap among Biological Aging Clocks: New Challenges in Biogerontology. Ageing Res. Rev. 2021, 72, 101502
4) Maisel et al. Self-reported Patient Motivations for Seeking Cosmetic Procedures. JAMA Dermatol 2018, 154, 1167-1174
5) Mohiuddin, A. (2019). Skin aging & modern age anti-aging strategies-global journals. Retrieved from https://globaljournals.org/GJMR_Volume19/3-Skin-Aging-and-Modern-Age.
6) Hawe et al, Genetic variation influencing DNA methylation provides insights into molecular mechanisms regulating genomic function. Nat Genet. 2022 January; 54 (1): 18-29. doi: 10.1038/s41588-021-00969-x
7) Ng and Chew. A systematic review of skin aging genes: gene pleiotropy and genes on the chromosomal band 16q24.3 may drive skin ageing. Sci Rep 12, 13099 (2022)
8) Villicaña, S., Bell, J. T. Genetic impacts on DNA methylation: research findings and future perspectives. Genome Biol 22, 127 (2021). https://doi.org/10.1186/s13059-021-02347-6

What is claimed is:

1. A method of predicting facial biological age of an individual, comprising:
a computer receiving methylation data describing an individual;
the computer receiving facial image data describing the individual;
the computer receiving survey data provided by the individual; and
the computer applying an age predictor clock model to at least the received data to predict a facial biological age of the individual.

2. The method of claim 1, further comprising the computer identifying causal methylation markers in the methylation data.

3. The method claim 1, further comprising the computer generating a personalized report for the individual, the report describing the predicted facial biological age based on methylation markers, and the report further describing methylation markers causal to facial aging, the markers identified at least in the data.

4. The method of claim 3, wherein the personalized report further contains facial ageotypes from face image data.

5. The method of claim 1, further comprising the computer copying the received data and the predicted facial biological age to a reference population database.

6. The method of claim 1, wherein the age predictor clock model is trained and validated on reference population data stored in the reference population database.

7. The method of claim 1, wherein face image data of the individual is one of a selfie image taken by a smartphone camera and an image captured by a professional imaging device.

8. The method of claim 7, wherein facial age-related phenotypes (ageotypes) are extracted, via a machine learning (AI) classifier, from the facial image data, wherein the classifier is one of a proprietary, an open-source, and a third-party algorithm utilized via an application programming interface (API).

9. A system for continual improvement of age prediction based at least on methylation data, comprising:
a computer and application executing thereon that:
receives epigenetics data containing at least DNA methylation markers describing an individual,
receives facial image data describing the individual,
receives feedback data and survey data comprising at least chronological age and gender of the individual,
predicts a facial biological age of the individual based on the data, and
propagates the received data and the predicted age to a reference population storage.

10. The system of claim 9, wherein the system uses the received data and previously stored data to improve a facial biological age prediction algorithm.

11. The system of claim 9, wherein the feedback data is further propagated to an age predictor engine and a reporter engine to improve a facial biological age prediction algorithm and identify methylation markers that are one of causal drivers of facial aging and causal anti-aging methylation markers.

12. The system of claim 9, wherein DNA methylation markers (CpGs) are pre-processed using bioinformatics methods directed to obtaining quantifiable results to enable further assessments.

13. The system of claim 9, wherein the system enables input of methylation data to compare facial biological ages of individuals before and after a recommended skincare treatment provided by at least a third party.

14. The system of claim 9, wherein the system builds predictive models for facial age-related phenotypes comprising at least one of wrinkles and pigmented spots, for skin age-related conditions comprising at least seborrheic keratosis, and for skin diseases comprising at least basal cell carcinoma.

15. A method for using methylation markers associated with ageotypes, comprising:
a computer applying epigenome-wide Mendelian Randomization (EWMR) to received data describing at least one individual;
the computer identifying, via the applied EWMR, methylation markers (CpGs) causal to at least one ageotype;
the computer utilizing epigenome-wide methylation (meQTL) data as exposure; and
the computer validating the identified methylation markers.

16. The method of claim 15, further comprising the computer validating markers using data from a reference population database.

17. The method of claim 15, further comprising the computer applying the EWMR to utilize summary statistics from genome-wide association studies for facial ageotypes as outcomes.

18. The method of claim 15, further comprising the computer observing and measuring facial ageotype and extracting ageotype from at least one of face image data, survey, and feedback data.

19. The method of claim 15, wherein epigenome-wide methylation data (meQTL) contain SNP-CpG associations detected in a biological sample comprising at least one of whole blood, skin, hair, and saliva.

20. The method of claim 15, wherein methylation markers (CpGs) associated with at least one facial ageotype are identified by one of correlative analyses and generalized linear regression from reference population data and wherein facial ageotype data are at least one of observable and measurable and are extracted from at least one of face image data, survey data, and feedback data.
